(19) United States
(12) Patent Application Publication
(10) Pub. No.: US 2017/0325794 A1
(43) Pub. Date: Nov. 16, 2017

(54) Endoscopic Suture And Cinch Anastomotic Leak Repair Device

(71) Applicant: BOSTON SCIENTIFIC SCIMED, INC., Maple Grove, MN (US)

(72) Inventors: Martin R. WILLARD, Burnsville, MN (US); Anthony F. TASSONI, JR., Andover, MN (US); Nicholas L. TASSONI, Andover, MN (US); Patrick A. HAVERKOST, Corcoran, MN (US); Gary J. PEDERSON, JR., Albertville, MN (US); Joseph M. CONNOLLY, Minneapolis, MN (US)

(21) Appl. No.: 15/595,710

(22) Filed: May 15, 2017

Related U.S. Application Data
(60) Provisional application No. 62/337,090, filed on May 16, 2016.

(51) Int. Cl. A61B 17/00; A61B 17/122; A61B 17/128; A61B 17/08

(52) U.S. Cl. CPC A61B 17/00234; A61B 17/083; A61B 17/1227; A61B 17/1285; A61B 17/0057; A61B 2017/00659; A61B 2017/00818; A61B 2017/00862; A61B 2017/00238

(57) ABSTRACT

A system for treating tissue includes a first fastener including a first anchoring element at a distal end thereof for anchoring the first fastener in a first target portion of tissue extending about a periphery of a tissue defect to be treated and a first suture extending from a proximal end thereof. The system also includes a second fastener including a second anchoring element at a distal end thereof for anchoring the second fastener in a second target portion of tissue extending about a periphery of a tissue defect to be treated and a second suture extending from a proximal end thereof. Furthermore, the system includes a cinch element disposed about both of the first and second sutures and slidable therealong such that a tension applied to the first and second sutures draws the first and second fasteners toward one another to close the tissue defect.

### PRIORITY CLAIM

The present disclosure claims priority to U.S. Provisional Patent Application Ser. No. 62/337,090 filed May 16, 2016; the disclosure of which is incorporated herewith by reference.

### BACKGROUND

Physicians have been increasingly willing to perform more aggressive interventional and therapeutic endoscopic procedures including, for example, the removal of larger lesions (e.g., cancerous masses), tunneling under the mucosal layer of the gastrointestinal (GI) tract to treat tissue below the mucosa, full thickness removal of tissue, the treatment of issues on other organs by penetrating the passing of the GI tract, and the endoscopic treatment/repair of post-surgical issues (e.g., post-surgical leaks, breakdowns of surgical staple lines, anastomotic leaks). These procedures may increase the risk of perforating the wall of the GI tract, or may require closure of the GI tract wall as part of the procedure. Endoscopic closure can save costs for the hospital and provide benefits to the patient. However, current devices for tissue closure are often difficult to use and time consuming. In addition, current devices may be insufficient to close certain perforations or to treat certain conditions and anatomies such as, for example, large wounds created in the GI tract.

### SUMMARY

The present disclosure is directed to a system for treating tissue, comprising a first fastener including a first anchoring element at a distal end thereof for anchoring the first fastener in a first target portion of tissue extending about a periphery of a tissue defect to be treated, a second fastener including a second anchoring element at a distal end thereof for anchoring the second fastener in a second target portion of tissue extending about the periphery of the tissue defect to be treated, a first suture coupled to a proximal end of the first fastener, a second suture coupled to a proximal end of the second fastener, and a cinch element disposed about both of the first and second sutures and slidable therealong such that a tension applied to the first and second sutures draws the first and second fasteners toward one another to close the tissue defect.

In an embodiment, the system may further comprise a third fastener including a third anchoring element at a distal end thereof for anchoring the third fastener in a third target portion of tissue about the periphery of the tissue defect and a third suture coupled to a proximal end of the third fastener.

In an embodiment, each of the first and second anchoring elements is one of a T-tag, a star hook, a barb, a hook, and a projection.

In an embodiment, the system may further comprise a first and a second locking element disposed about the first and second sutures, respectively, and slidable therealong to secure the first and second fasteners to the first and second target portions of tissue, respectively.

In an embodiment, the system may further comprise a delivery tool including a longitudinal sheath extending longitudinally from a proximal end to a distal end and including a lumen extending therethrough, the lumen sized and shaped to movably receive a mandrel longitudinally therein.

In an embodiment, movement of the mandrel distally within the sheath moves each of the first and second locking elements distally along the first and second sutures, respectively.

In an embodiment, the delivery tool further comprises a push tube for advancing cinch element distally along the first and second sutures.

In an embodiment, the cinch element is one of a cinch knot and a cinch washer.

In an embodiment, the longitudinal sheath is configured to push each of the first and second fasteners, located at a distal end thereof, through the first and second target portions of tissue, respectively.

The present disclosure is also directed to a system for treating a tissue defect, comprising a delivery sheath extending from a proximal end to a distal end and including a plurality of lumens extending therethrough, a first fastener including a first suture extending from a proximal end thereof and a first anchoring element at a distal end thereof for anchoring the first fastener in a first target portion of tissue, the first fastener located at a distal end of a first one of the plurality of lumens, a second fastener including a second suture extending from a proximal end thereof and a second anchoring element at a distal end thereof for anchoring the second fastener in a second target portion of tissue, the second fastener located at a distal end of a second one of the plurality of lumens, and a cinch element disposed about the first and second sutures and slidable therealong such that tension applied to the sutures draws the first and second fasteners toward one another to close the tissue defect.

In an embodiment, the system may further comprise a third fastener including third suture extending from a proximal end thereof and a third anchoring element at a distal end thereof for anchoring the third fastener in a third target portion of tissue about the periphery of the tissue defect, the third fastener located at a distal end of a third one of the plurality of lumens.

In an embodiment, each of the first and second anchoring elements is one of a T-tag, a star hook, a barb, a hook, and a projection.

In an embodiment, the system may further comprise a first and a second locking element disposed about the first and second sutures and slidable therealong to secure the first and second fasteners to the first and second target portions of tissue.

In an embodiment, the system may further comprise a mandrel configured to be movably received within each the first, second and third lumens, movement of the mandrel distally within the sheath moving each of the first, second and third locking elements distally along first, second and third sutures, respectively.

In an embodiment, the system may further comprise a push tube for advancing cinch element distally along the first and second sutures.

The present disclosure also relates to a method for treating a tissue defect, comprising placing a first fastener in a first target portion of tissue about a periphery of a tissue defect to be treated, the first fastener including a first suture extending from a proximal end thereof, placing a second fastener in a second target portion of tissue about a periphery of a tissue defect to be treated, the second fastener including a second suture extending from a proximal end thereof, and sliding a cinch element distally along the first and second sutures to draw the first and second fasteners toward one another to close the tissue defect.

### BRIEF DESCRIPTION

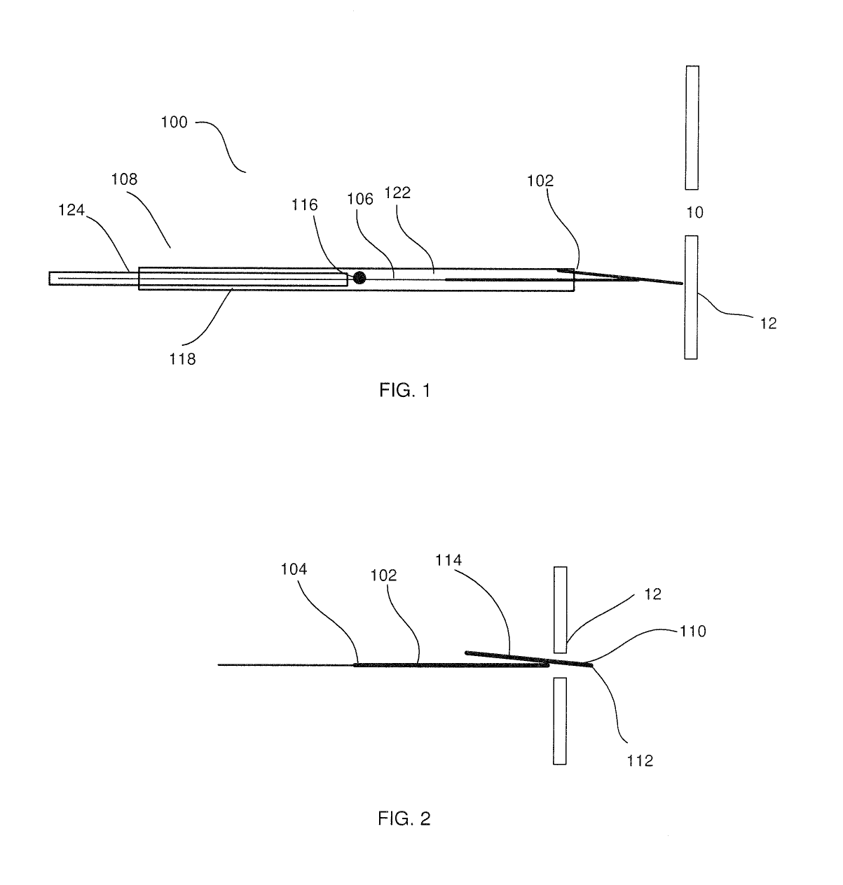

FIG. 1 shows a side view of a system according to a first exemplary embodiment of the disclosure in a first configuration;

FIG. 2 shows a side view of a fastener of the system of FIG. 1 in a first configuration;

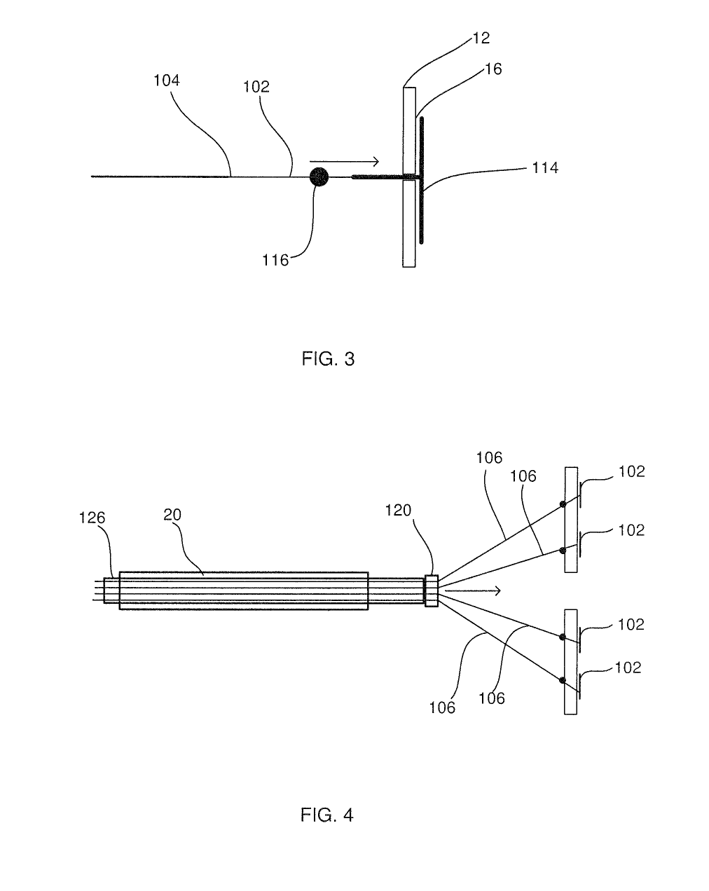

FIG. 3 shows another side view of the fastener of the system of FIG. 1 in a second configuration;

FIG. 4 shows a side view of the system of FIG. 1 in a second configuration;

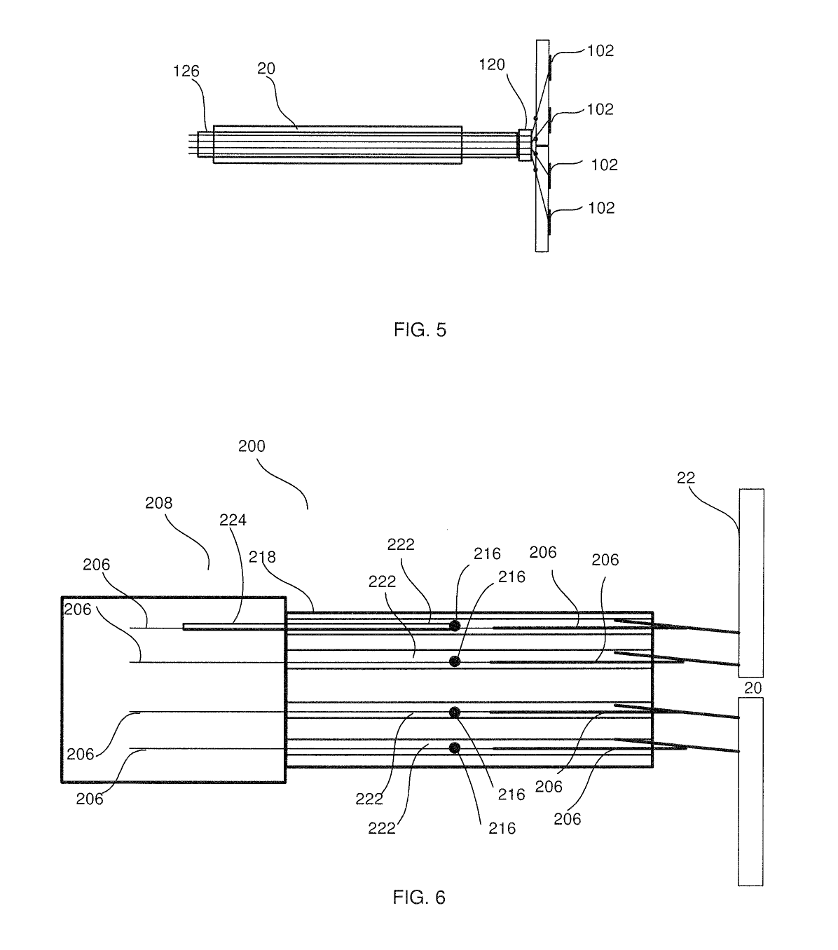

FIG. 5 shows a side view of the system of FIG. 1 in a third configuration;

FIG. 6 shows a side view of a system according to a second exemplary embodiment of the present disclosure in a first configuration;

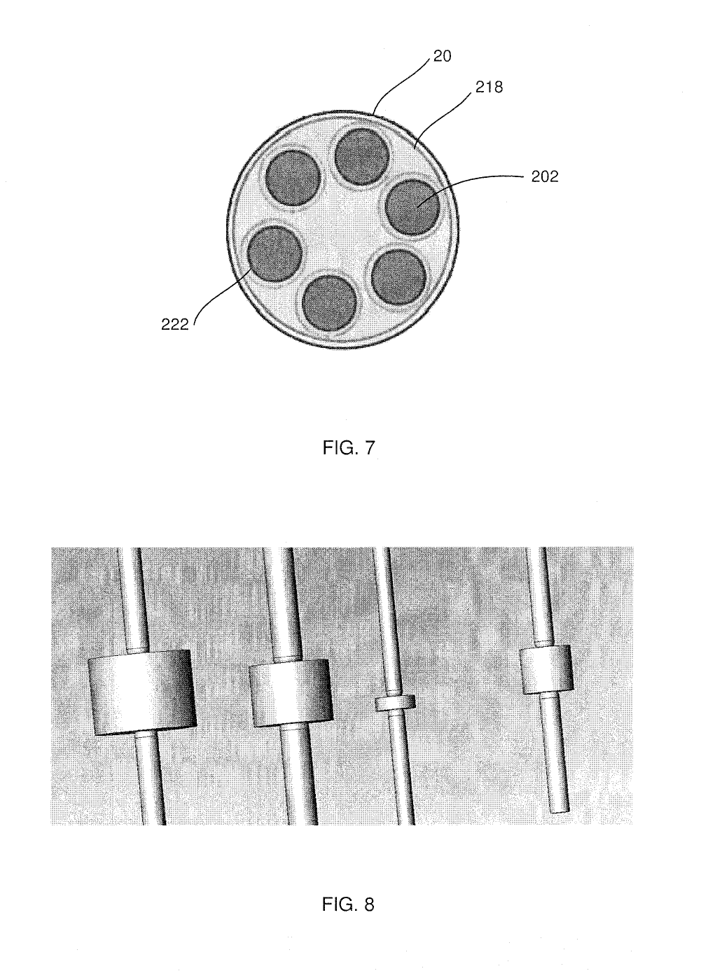

FIG. 7 shows a cross-sectional view of the system of FIG. 6;

FIG. 8 shows a perspective side view of locking elements of an exemplary embodiment of the system.

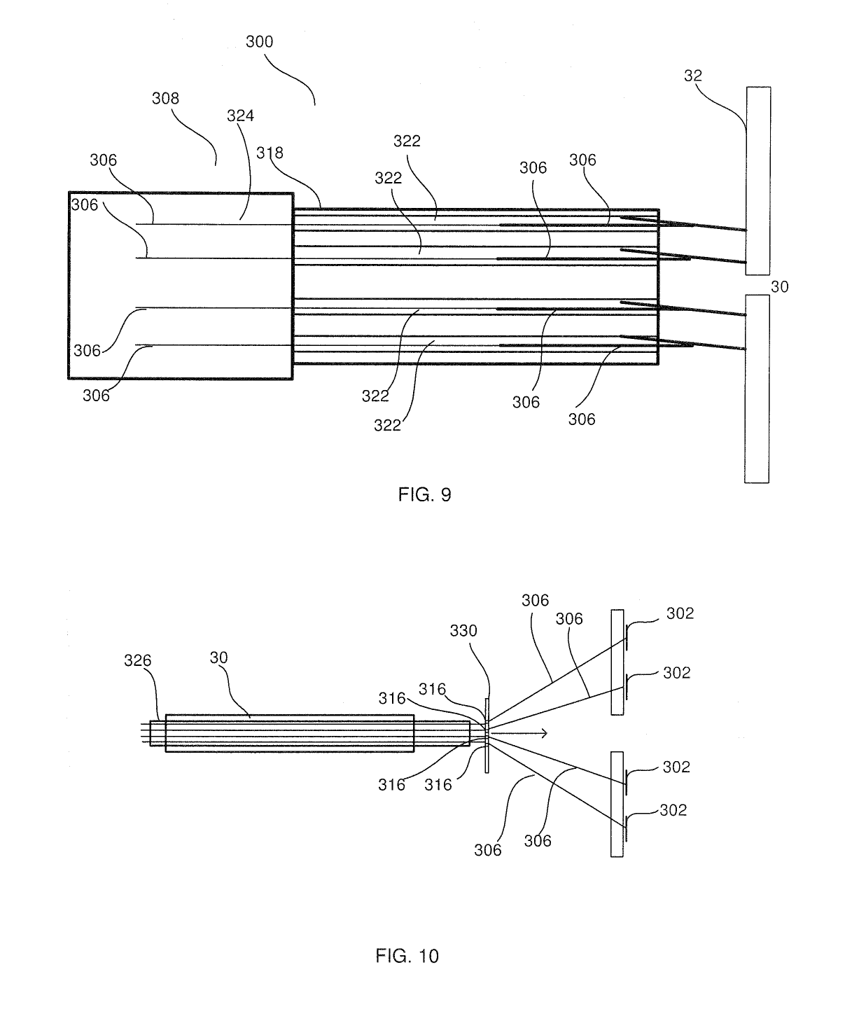

FIG. 9 shows a side view of a system according to a second exemplary embodiment of the present disclosure in a first configuration;

FIG. 10 shows a side view of the system of FIG. 9 in a second configuration;

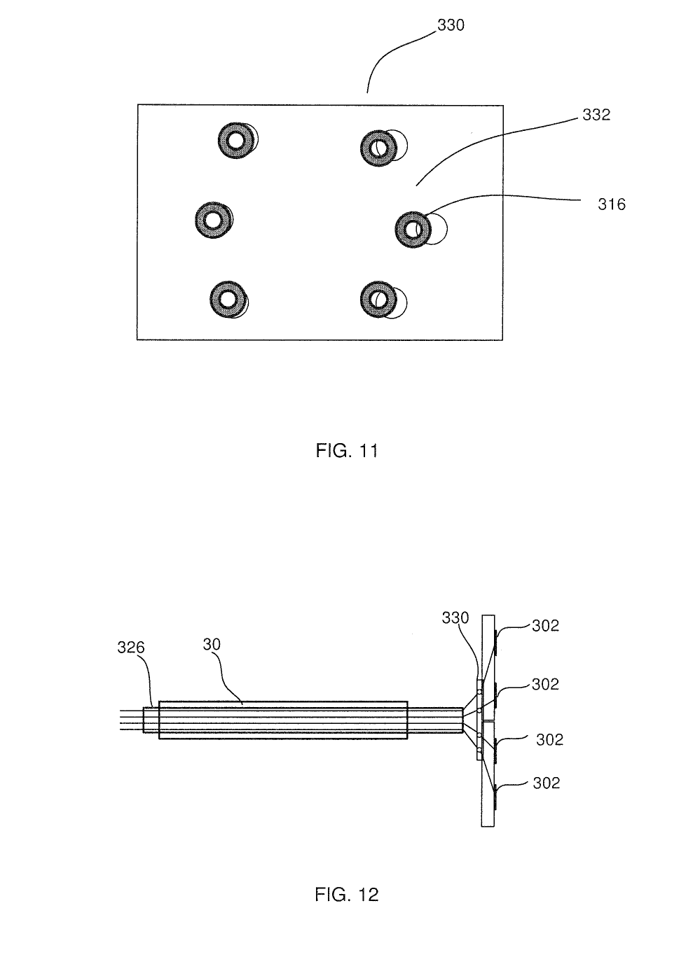

FIG. 11 shows a cross-sectional view of the collagen graft piece of the system of FIG. 9; and

FIG. 12 shows a side view of the system of FIG. 9 in a third configuration.

### DETAILED DESCRIPTION

The present disclosure may be further understood with reference to the following description and the appended drawings, wherein like elements are referred to with the same reference numerals. The present disclosure is directed to devices for the treatment of tissue and, in particular endoscopic tissue treatment devices. Exemplary embodiments of the present disclosure describe fasteners positioned about a periphery of a tissue opening and drawn toward one another via sutures to close the tissue opening. It should be noted that the terms “proximal” and “distal” are intended to refer to a direction toward (proximal) and away from (distal) a user of the device.

As shown in FIGS. 1-5, a system 100 according to a first exemplary embodiment of the present disclosure comprises a plurality of fasteners 102 with each of the fasteners 102 configured to be inserted into target tissue locations distributed around a periphery of a tissue opening 10. A suture 106 extends proximally from a proximal end 104 of each of the fasteners 102. Thus, after insertion of a desired number of fasteners 102 into tissue around the tissue opening 10, the sutures may be drawn together to pull the fasteners 102 toward one another, drawing the edges of the tissue opening 10 toward one another to close the tissue opening. The sutures 106 may then be locked in this position (e.g., by a cinch mechanism) to hold the opening 10 closed. The fasteners 102 may be delivered to a target tissue 12 about the tissue opening 10 via a delivery tool 108 formed as a tube sized and shaped to be inserted through, for example, a working channel of an endoscope 20.

Each fastener 102 extends from a proximal end 104 to a distal end 110. The distal end 110 includes a distal tip 112 configured (e.g., sharpened) for piercing target tissue into which the fastener 102 is to be inserted. The distal end 110 also includes an anchoring element 114 such as a T-tag 114 which prevents the distal end 110 from disengaging from tissue into which it has been inserted. The T-tag 114 may be biased toward a T-shaped configuration in which the T-tag 114 extends transverse (e.g. perpendicularly) to a length of a proximal portion of the fastener 102, as shown in FIGS. 2-3. During insertion, the T-tag 114 is constrained by the delivery tool 108 to remain in an insertion configuration in which extends substantially parallel a longitudinal axis of the fastener 102, as will be described in further detail below. In the insertion configuration, the T-tag 114 passes more easily through an insertion device and into target tissue 12 via the sharpened distal tip 112. Once the fastener 102 has been inserted distally through the target tissue 12 with the T-tag 114 extending distally past a distal surface 16 of the target tissue 12, the T-tag 114 reverts under its bias to its T-shaped configuration with the T-tag 114 extending lateral to the path along it passed through the target tissue 12 to hold the fastener 102 in position within the target tissue 12. The fastener 102 may include a biodegradable material so that, over time, the anchor 114 is degraded, eroded, and/or absorbed into the body. In some cases, the fastener 102 may include a PLGA, PLLA, PGA or other degradable or erodible polymers, such as polyesters, polysaccharides, polyanhydrides, polycaprolactone and various combinations thereof. In some cases, the fastener 102 may include a combination of the previously mentioned materials to impart a variable strength and/or degradation time profile in the fastener 102. It is understood that the anchoring element 114 of the fastener 102 may be star shaped, a hook, a barb, a projection, or any other configuration that enables the fastener 102 to anchor itself within the target tissue 12.

Each suture 106 extends through the delivery tool 108 to a proximal end (not shown) which, during use, remains outside the patient's body accessible to the user of the device. Any or all of the fasteners 102 may include an eyelet (not shown) or other structure to facilitate attachment of the distal end of the suture 106 to the fastener 102. As would be understood by those skilled in the art, the suture 106 may be formed of a biodegradable material so that, over time, the suture 106 degrades and is absorbed into the body. The suture 106 may include a PLGA, PLLA, PGA or other degradable or erodible polymers, such as polyesters, polysaccharides, polyanhydrides, polycaprolactone, and various combinations thereof.

As shown in FIGS. 3-4, one or more locking elements 116 may be used to secure the fasteners 102 relative to the target tissue 12. The locking element 116 may be disposed about at least a portion of the suture 106 proximal to the fastener 102. The locking elements 116 are configured to slide over the suture 106 to a desired position near the target tissue 12 to hold each fastener 102 in place within the target tissue 12—i.e., to prevent the fasteners 102 from passing distally entirely through the tissue. In an exemplary embodiment, the locking element 116 is slidable distally over the suture 106 to a position abutting the target tissue 12, maintaining the distal portion of the suture 106 under tension. In an embodiment, the locking element 116 can be a knot, such as a compression knot that may exert a radial force on the suture 106. The knot may be a rolling hitch, safety belt hitch, blake's hitch or any other knot known in the field of art. As would be understood by those skilled in the art, the knot may be formed to have a friction force resisting movement relative to the suture 106 of 0.5 pounds, 1 pound, 1.5 pounds, 2.0 pounds, 2.5 pounds, 3.0 pounds, or other suitable friction force depending on the form of the knot 116. The friction force exerted by the knot 116 is preferably greater than a rebound force of the tissue 12 against which it is abutting to prevent the knot 116 from moving so that the fastener 102 is held in place within the target tissue 12. In an alternative embodiment, no locking mechanism 116 is used and the fasteners 102 are held in place by the cinch element 120, or cinch knot described below.

In a further embodiment, the locking element 116 may be separate and independent from the suture 106. For example, the locking element 116 may include a filament that is independent from the suture 106. In an example, the filament of the locking element 116 has a radial diameter larger than that of the suture 106 so that a size of the locking element 116 is sufficient to distribute a desired amount of force against the proximal side of the target tissue 12 without penetrating into the tissue 12.

In another embodiment, the locking element 116 is a sliding washer, a disc shaped retainer, or other device. In an example, the locking element 116 may be moved to a configuration permitting it to slide relative to the suture 106 when subject to an exertion of force and, when this force is released, the locking element will revert to a locking configuration preventing movement relative to the suture 106. For example, an opening in the locking element 116 may be formed so that, when the locking element 116 is bent away from a shape to which it is biased, the opening enlarges. Thus, when the locking element 116 is no longer subject to the external force, it reverts to its natural shape closing the opening against the suture and locking the position of the locking element 116 relative to the suture 106. In another example, the locking element 116 is configured to slide distally over the suture 106 but is prevented by the structure of an opening therein from sliding proximally relative to the suture 106. In another example the locking element 116 may be formed as a cinch that may be crushed or otherwise deformed when it has reached the desired position to prevent relative movement between the locking element 116 and the suture 106. In yet a further embodiment, the locking element 116 may be formed with an opening that slides over the suture 106 when the suture 106 is under tension. That is, the suture 106 may be drawn under tension so that its diameter is smaller while under tension permitting the locking element 116 to slide freely over the suture 106. Then, when tension is released from the suture 106, the suture 106 reverts to its larger unstressed diameter locking the locking element 116 in place. The locking element 116 thickness may vary according to a preferred locking force, as can be seen in FIG. 8. For example, increasing the locking element 116 thickness will increase the locking force on the suture 106 while decreasing the locking element 116 thickness will decrease the locking force on the suture 106.

The locking elements 116 may also be formed of a biodegradable material so that, over time, the locking element 116 is degraded, eroded and/or absorbed by the body. In some cases, the locking element 116 may include PLGA, PLLA, PGA or other degradable or erodible polymers, such as polyesters, polysaccharides, polyanhydrides, polycaprolactone, and various combinations thereof.

After the fasteners 102 have been positioned within the target tissue 12 around the locking elements 116 have been configured to hold each of the fasteners 102 around the tissue opening 10 as desired, proximal ends of the sutures 106 may be passed through a cinch element 120 which is pushed distally over the sutures 102 to draw the sutures 106 together pulling the fasteners 102 and the edges of the tissue opening 10 toward one another to close the opening. Once the cinch member 120 has reached the desired position in which the opening 10 is held closed, the cinch member 120 is locked in position relative to the sutures 106 to maintain the opening 10 closed. For example, the cinch member 120 may be crushed over the sutures 106, or, as would be understood by those skilled in the art, may include a structure such as a pinch into which the sutures 106 may be drawn and locked in position. Alternatively, as would be understood by those skilled in the art, a cinch knot may be formed around the sutures 106 and pushed distally along the sutures 106 to a desired position closing the opening 10. The knot may then be tightened to lock the opening 10 closed. In an embodiment, the knot may be pre-tied around all of the sutures 106 before placement of the fastener 102 within the target tissue. In another embodiment, the cinch knot 120 may be mounted around all of the sutures 106 after placement of the fasteners 102 within the target tissue 12. The cinch knot 120 may be configured to slide distally over the sutures 106 held therein to draw the fasteners 102 and the edges of the tissue opening 10 toward one another, closing the tissue opening 10. The cinch knot 120 may be a rolling hitch, safety belt hitch, blake's hitch or any other knot known in the field of art.

Similar to the locking element 116, the cinch knot 120 may be a compression knot that may exert a radial force on the sutures 106. As would be understood, the cinch knot 120 may be tightened to the degree necessary to resist the force applied by the surrounding tissue that would tend to reopen the opening 10. The cinch knot 120 may be separate and independent from the sutures 106. The cinch knot 120 may include a filament that is independent of the sutures 106.

In one exemplary embodiment, a cinch button/washer 120′ may be used. The cinch button 120′ may be a disc shaped retainer with an opening therethrough through which the sutures 106 pass. The cinch button 120′ is formed so that the opening receives the sutures 106 loosely when the cinch button 120′ is subjected to a deforming force that increases a size of the opening. In an example, the cinch button 120′ may be configured to slide distally over the sutures 106 while deformed within an insertion device. After being moved distally out of the insertion device, the cinch button 120′ may revert to a resting shape under a natural bias which closes the opening preventing the cinch button 120′ from moving proximally over the sutures 106. As described above, tension may also be placed on the sutures 106 to reduce their diameters, allowing the sliding cinch button 120′ to slide through the cinch button 120′. Then, when the cinch button 120′ reaches its desired position, the tension on the sutures 106 is released permitting the sutures 106 to expand to their starting diameters locking the cinch button 120′ in place through the friction applied by the expanded sutures 106.

The cinch element 120 may be formed of a biodegradable material so that, over time, the cinch knot is degraded, eroded, and/or absorbed into the body. In some cases, the cinch knot/button may include a PLGA, PLLA, PGA or other degradable or erodible polymers, such as polyesters, polysaccharides, polyanhydrides, polycaprolactone, and various combinations thereof.

As shown in FIG. 1, a flexible delivery tool 108 may include a longitudinal delivery catheter or sheath 118 capable of passing through body lumens such as those traversed, for example, by flexible endoscopes. The sheath 118 extends longitudinally from a proximal end to a distal end and includes a lumen 122 extending therethrough. A first fastener 102 is disposed at a distal end of the lumen 122, either within the sheath 118 or partially within the sheath 118. The suture 106 and locking element 116 may also be disposed within the sheath 118. A push member or mandrel 124 is slidably received in the lumen 122 to contact the proximal end of the locking element 116 to push the fastener 102 distally through the lumen 122. Movement of the mandrel 124 distally within the delivery sheath 118 moves the locking element 116 and the fastener 102 distally through the lumen 122 until the fastener 102 projects distally from the delivery tool 108 and penetrates the target tissue 12 at a desired location. The push member 124 is moved distally until the T-tag 114 reaches the distal side of the target tissue 12 and reverts to its T-shaped configuration. The locking element 116 is moved distally along the suture 106 and locked in place to hold the fastener 102 at the desired location in the target tissue 12 and the same process is repeated with additional fasteners 102 until a desired number of fasteners 102 have been placed around the opening 10 to be closed, as can be seen in FIGS. 2-4. The proximal ends of the sutures 106 are then passed through the cinch element 120 which is slid through the deliver tool 108 until the tissue surrounding the opening 10 is drawn together as desired sealing the opening 10. The cinch element 120 is then locked in the desired position, holding the opening 10 sealed.

According to an exemplary method using the system 100, the delivery tool 108, including the fastener 102, suture 106, and locking element 116 housed within the lumen 122, is inserted into a body to target tissue 12 therein via, for example, a working channel of an endoscope or other insertion device. The delivery tool 108 is then positioned over the target tissue 12 such that the target tissue 12 (e.g. tissue along a periphery of the tissue opening 10 to be closed) is adjacent the sharp distal end 110 of the fastener 102 and push member 124 is moved distally to push the fastener 102 through the tissue. The distal tip 110 of the fastener 102 pierces the target tissue 12, extending distally past the distal surface 16 of the target tissue 12, allowing the T-tag to revert to its biased T-shaped configuration to hold the fastener 102 in position within the target tissue 12.

Once the fastener 102 is positioned within the target tissue 12, the mandrel 124 may be moved distally within the delivery sheath 118 to slide the locking element 116 distally down the suture 106 attached to the proximal end of the fastener 102. The mandrel 124 may be advanced against the locking element 116, as shown in FIG. 1, until the locking element is positioned against a proximal side of the target tissue 12, aiding in holding the fastener 102 in place within the target tissue 12. Proximal and distal movement of the mandrel 124 may be facilitated manually or via an actuation mechanism, as would be understood by those skilled in the art. The delivery sheath 118 is then removed, leaving the singular suture 106 and fastener 102 in position around the target tissue 12. Withdrawal of the delivery sheath 118 may be facilitated manually or by an actuation mechanism housed within the delivery tool 108.

As shown in FIG. 4, subsequent fasteners 102 may be inserted through the proximal end of the delivery tool 108 and advanced into desired locations in the target tissue 12 in the same manner until the desired number of fasteners 102 have been positioned as required to seal the opening 10. Then, the proximal ends of the sutures 106 are passed through the cinch element 120 which is pushed through the delivery tool 108 and slid distally along the sutures 106 to a desired position in which the tissue surround the opening 10 is drawn together to seal the opening 10. The cinch element 120 may be slid along the sutures 106 manually or by the push tube 126 to close the tissue opening 10. The cinch element 120 (or cinch knot) is then locked in the desired position and the push tube 126 and delivery tool 108 are withdrawn proximally, leaving the fasteners 102 and sutures 106 fixed to the target tissue 12. Excess suture may then be trimmed as desired.

As shown in FIGS. 6-8, a system 200 according to another exemplary embodiment may be substantially similar to the system 100, comprising a plurality of fasteners 202 to be placed in target tissue 22 about a periphery of a tissue opening 20 via a delivery tool 208. The fasteners 202 may be substantially similar to the fasteners 102.

The delivery tool 208 may also be substantially similar to the delivery tool 108 except that, instead of a single lumen delivery tube that handles only a single fastener at a time, the delivery tool 208 is formed as a tube that slidably receives therein a delivery sheath 218 having multiple lumens for the delivery of multiple fasteners 202 without requiring each fastener 202 to be loaded individually into the delivery tool 208 after a previous fastener 202 has been placed as desired. As shown in FIG. 7, the delivery sheath 218 includes a plurality of lumens 222, with each lumen 222 including a fastener 202 disposed therein. In an exemplary embodiment, the delivery sheath 218 includes 6 lumens. However, those skilled in the art will understand that the delivery sheath 218 may include two or more lumens to any desired number of fasteners 202 to be placed around an opening 10. As with the previous embodiment, each fastener 202 is coupled to a suture 206 and may include a locking element 216 all of which may reside in the corresponding lumen 222. In an embodiment, a single push member or mandrel 224 may be inserted slidably into any desired one of the lumens 222 to distally slide the locking element 216 of a desired fastener 202 to deploy the desired fastener 202 in the same manner described above for the fasteners 102. The sheath 218 may then be repositioned to align another one of the lumens 222 and the corresponding fastener 202 with a second desired location at which this fastener 202 is to be deployed. The process is then repeated until all of the desired fasteners 202 have been positioned as desired. In another embodiment, each lumen 222 may include a multi-pronged mandrel 224 including a prong for each lumen 222 (or for any desired number of the lumens 222) to deploy multiple fasteners 202 at the same time. In this embodiment, as would be understood by those skilled in the art, the positioning of these fasteners 202 relative to one another is determined by the spacing between the lumens 222. As described above, in a further embodiment, no locking element 216 is used to secure the fasteners 202 within the target tissue 22.

Then, when all the desired fasteners 202 have been placed in the target tissue 22 as desired, the delivery sheath 218 is slid proximally over the sutures 206 through the delivery tool 208 and withdrawn from the body. When sutures 206 have been fully withdrawn from the delivery sheath 218, the sutures 206 may be locked in the desired position in the same manner described above for the sutures 106 to close the opening as desired.

Substantially similar to system 100, cinch element 220 may then be positioned about a proximal portion of the sutures 206 so that each of the sutures 206 is held within the cinch element 220. Positioning of the cinch element 220 may be facilitated manually or by any other appropriate means, as would be understood by those skilled in the art. The cinch element 220 is then slid distally along the sutures 206, drawing the fasteners 202 toward one another and holding the sutures 206 in a tightened configuration. As with system 100, the cinch element 220 may be slid along the sutures 206 manually or by push tube 226 to close the tissue opening 10. If a push tube is used, the push tube 226 is then withdrawn proximally, leaving the cinch element 220, fasteners 202 and sutures 206 fixed to the target tissue 22. Sutures 206 are then released from the delivery tool 208 and the delivery tool 208 is removed from the body. Portions of the sutures 206 extending proximally from the cinch element 220 may then be cut or otherwise disposed of as would be understood by those skilled in the art.

As shown in FIGS. 9-12, a system 300 is substantially similar to the system 200 described above, and comprises a plurality of fasteners 302 configured to engage tissue. The fasteners 302 may be delivered to target tissue via a delivery tool 308 substantially similar to delivery tool 208 described above. Rather than a cinch knot or cinch button, however, a collagen graft piece 330 may be used, as shown in FIG. 11. The collagen graft piece 330 may include a pre-selected hole punch pattern 332 through which the sutures 306 may be weaved to move the target tissue 32 to a desired pre-selected closed configuration. The collagen graft piece 330 may be any shape so as to conform to the target tissue 32 and opening 30. The collagen graft piece 330 may include locking elements 316 for suture 306 incorporated therein such that as the graft piece 330 is slide distally along the plurality of sutures 306, the graft piece 330 both aids in holding the fasteners 302 in place within the target tissue 32 and pulls the sutures 306 closer together so as to pull the fasteners 302 closer together, closing the tissue opening 30. In an embodiment, the sutures 306 may be pre-strung through the collagen graft piece 330.

According to an exemplary method, the delivery tool 308, including the plurality of fasteners 302 and sutures 306 housed within the plurality of lumens 322 of the delivery sheath 318 is inserted into a working channel of an endoscope or other insertion device. The fasteners 302 may be pressed simultaneously through the tissue via the delivery sheath 318 so that the fasteners 302 pierce the tissue. Once the fasteners 302 are positioned within the target tissue 32, the delivery sheath 318 is removed either manually or by an actuation mechanism housed within the delivery tool 308, leaving the sutures 306 and fasteners 302 in position around the target tissue 32. Sutures 306 may then be threaded through the collagen graft piece 330 through the pre-selected hole punch pattern 332. The collagen graft piece 330 is then slid distally along the sutures 306, drawing the fasteners 302 toward one another to close the tissue opening 30. The collagen graft piece 330 may be moved either manually or by push tube 326. If a push tube 326 is used, the push tube 326 is then withdrawn proximally, leaving the collagen graft piece 330, sutures 306 and fasteners 302 fixed to the target tissue. Sutures 306 are then released from the delivery tool 308 and the delivery tool 308 removed from the tissue tract.

It will be apparent to those skilled in the art that various modifications may be made in the present disclosure, without departing from the scope of the disclosure. Thus, it is intended that the present disclosure cover modifications and variations of this disclosure provided that they come within the scope of the appended claims and their equivalents.

1-15. (canceled)

16. A system for treating tissue, comprising:
a first fastener including a first anchoring element at a distal end thereof for anchoring the first fastener in a first target portion of tissue extending about a periphery of a tissue defect to be treated;
a second fastener including a second anchoring element at a distal end thereof for anchoring the second fastener in a second target portion of tissue extending about the periphery of the tissue defect to be treated;
a first suture coupled to a proximal end of the first fastener;
a second suture coupled to a proximal end of the second fastener; and
a cinch element disposed about both of the first and second sutures and slidable therealong such that a tension applied to the first and second sutures draws the first and second fasteners toward one another to close the tissue defect.

17. The system of claim 16, further comprising a third fastener including a third anchoring element at a distal end thereof for anchoring the third fastener in a third target portion of tissue about the periphery of the tissue defect and a third suture coupled to a proximal end of the third fastener.

18. The system of claim 16, wherein each of the first and second anchoring elements is one of a T-tag, a star hook, a barb, a hook, and a projection.

19. The system of claim 16, further comprising a first and a second locking element disposed about the first and second sutures, respectively, and slidable therealong to secure the first and second fasteners to the first and second target portions of tissue, respectively.

20. The system of claim 16, further comprising a delivery tool including a longitudinal sheath extending longitudinally from a proximal end to a distal end and including a lumen extending therethrough, the lumen sized and shaped to movably receive a mandrel longitudinally therein.

21. The system of claim 20, wherein movement of the mandrel distally within the sheath moves each of the first and second locking elements distally along the first and second sutures, respectively.

22. The system of claim 20, wherein the delivery tool further comprises a push tube for advancing cinch element distally along the first and second sutures.

23. The system of claim 16, wherein the cinch element is one of a cinch knot and a cinch washer.

24. The system of claim 16, wherein the longitudinal sheath is configured to push each of the first and second fasteners, located at a distal end thereof, through the first and second target portions of tissue, respectively.

25. A system for treating a tissue defect, comprising:
a delivery sheath extending from a proximal end to a distal end and including a plurality of lumens extending therethrough;
a first fastener including a first suture extending from a proximal end thereof and a first anchoring element at a distal end thereof for anchoring the first fastener in a first target portion of tissue, the first fastener located at a distal end of a first one of the plurality of lumens;
a second fastener including a second suture extending from a proximal end thereof and a second anchoring element at a distal end thereof for anchoring the second fastener in a second target portion of tissue, the second fastener located at a distal end of a second one of the plurality of lumens; and
a cinch element disposed about the first and second sutures and slidable therealong such that tension applied to the sutures draws the first and second fasteners toward one another to close the tissue defect.

26. The system of claim 25, further comprising a third fastener including third suture extending from a proximal end thereof and a third anchoring element at a distal end thereof for anchoring the third fastener in a third target portion of tissue about the periphery of the tissue defect, the third fastener located at a distal end of a third one of the plurality of lumens.

27. The system of claim 25, wherein each of the first and second anchoring elements is one of a T-tag, a star hook, a barb, a hook, and a projection.

28. The system of claim 25, further comprising a first and a second locking element disposed about the first and second sutures and slidable therealong to secure the first and second fasteners to the first and second target portions of tissue.

29. The system of claim 26, further comprising a mandrel configured to be movably received within each the first, second and third lumens, movement of the mandrel distally within the sheath moving each of the first, second and third locking elements distally along first, second and third sutures, respectively.

30. The system of claim 25, further comprising a push tube for advancing cinch element distally along the first and second sutures.

31. A method for treating a tissue defect, comprising:
placing a first fastener in a first target portion of tissue about a periphery of a tissue defect to be treated, the first fastener including a first suture extending from a proximal end thereof;
placing a second fastener in a second target portion of tissue about a periphery of a tissue defect to be treated, the second fastener including a second suture extending from a proximal end thereof; and
sliding a cinch element distally along the first and second sutures to draw the first and second fasteners toward one another to close the tissue defect.

32. The method of claim 31, further comprising placing a third fastener in a third target portion of tissue about a periphery of a tissue defect to be treated, the third fastener including a third suture extending from a proximal end thereof.

33. The method of claim 31, wherein the first and second anchoring devices are anchored to the first and second target portions of tissue via one of a T-tag, a star hook, a barb, a hook, and a projection.

34. The method of claim 31, further comprising:
inserting a delivery tool to the first target portion of tissue, a longitudinal member of the delivery tool including the first fastener at a distal end thereof;
moving the longitudinal member distally to push the first fastener through the first target portion of tissue;
moving a mandrel distally within the longitudinal member to slide a first locking element disposed about the first suture distally to secure the first fastener to the first target portion of tissue; and
withdrawing the longitudinal member proximally from the first target portion of tissue.

35. The method of claim 34, further comprising:
moving the longitudinal member of the delivery tool to the second target portion of tissue, the longitudinal member including the second fastener at a distal end thereof;
moving the longitudinal member distally to push the second fastener through the second target portion of tissue;
moving the mandrel distally within the longitudinal member to slide a second locking element disposed about the second suture distally to secure the second fastener to the second target portion of tissue; and
withdrawing the longitudinal member proximally from the second target portion of tissue.
